(19) United States
(12) Patent Application Publication
(10) Pub. No.: US 2011/0105822 A1
(43) Pub. Date: May 5, 2011

(54) BALLOON CATHETER AND X-RAY APPLICATOR COMPRISING A BALLOON CATHETER

(75) Inventor: Norman Roeder, Aalen (DE)

(73) Assignee: CARL ZEISS SURGICAL GMBH, Oberkochen (DE)

(21) Appl. No.: 12/989,814

(22) PCT Filed: Apr. 23, 2009
(86) PCT No.: PCT/EP09/02955

§ 371 Date: Dec. 22, 2010

(30) Foreign Application Priority Data
Apr. 30, 2008 (DE) 102008021733.6
Aug. 15, 2008 (DE) 102008041286.4

(51) Int. Cl. A61N 5/10; A61N 5/00

(52) U.S. Cl. 600/1; 378/65

(57) ABSTRACT

The invention relates to a balloon catheter for an X-ray applicator and an X-ray applicator—for use with the corresponding balloon catheter. Said balloon catheter can be filled with a medium and comprises a balloon—that expands with respect to the volume and a catheter shaft for inserting the X-ray applicator. Said balloon or the catheter shaft comprises a rigid inner end piece in the extension of the catheter shaft.

The present invention relates to a balloon catheter and an X-ray applicator with a balloon catheter.

An X-ray applicator with a balloon catheter for radiation therapy is described in U.S. Pat. No. 5,621,780 A.

There are substantially two methods used for the radiation therapy of tumors:

Irradiating the tumor by therapeutic radiation from a radiation source located outside of the patient and irradiating by means of a radiation source that has been introduced into the patient.

Radiation sources that can be inserted into a patient allow intraoperative therapy of patients by means of X-ray radiation. This method of treatment is referred to as intraoperative radiation therapy (IORT).

There are various methods in IORT for irradiating a tumor or a tumor bed from the inside. An advantageous method consists of establishing an access into the center of the tumor or the remaining tumor bed if the tumor was removed. This access can be brought about by means of applicators that, particularly during the irradiation of a tumor bed, also function as a placeholder: in this case the applicator is used to bring the dimensionally unstable tumor bed into a defined shape, preferably a spherical shape. This ensures uniform irradiation of the tissue surrounding the applicator. Point radiation sources are particularly suitable for this method of radiation therapy. The radiation therapy system INTRABEAM® by Carl Zeiss comprises such a point radiation source in the form of a source for X-ray radiation. This radiation therapy system comprises a probe that is approximately 10 cm long and only 3.2 mm wide, an X-ray probe, in which electrons are accelerated and decelerated on a target material. At a distal end of the X-ray probe, this generates X-ray radiation with spherical and isotropic emission characteristics. The radiation therapy system INTRABEAM® comprises various applicators into which the probe can be inserted and at the distal end of which X-ray radiation is generated.

Radiation therapy systems with different applicator embodiments are known in the art. A distinction is made between rigid and flexible applicators: rigid applicators are advantageous in terms of high dimensional accuracy and high dimensional stability. A very precise positional stop for a corresponding X-ray probe can be formed in a simple manner in these applicators. A disadvantage of these rigid applicators is that they cannot remain within the patient for a long period of time for wound-healing and handling reasons. Hence the use of such applicators is limited to irradiation performed directly after lumpectomy. In the process, the surgical access originally formed for the tumor extraction is used for irradiation by means of a corresponding applicator.

Catheters in particular are known as flexible applicators. Such a catheter is described in e.g. WO 2006/041733 A2. A biopsy channel is laid to the tumor bed for the purpose of radiation therapy using these applicators. In certain medical circumstances, the tumor can be removed directly through the catheter via the biopsy channel. Medical instruments can be inserted once, or else repeatedly, into the patient through the access to the tumor tissue created by the catheter in order thus to irradiate the tissue surrounding a tumor bed from the inside directly after a surgical operation or even at a later stage. Within the scope of therapy, such irradiation is performed once, or else more frequently, over a period of a number of days. So-called balloon catheters are particularly suitable catheters for this. Balloon catheters are tube-like structures that comprise one or more balloons that can be inflated and are formed at the distal end of the arrangement.

The catheter known from WO 2006/041733 A2 is e.g. such a balloon catheter. It is guided to the tumor bed through the biopsy channel. In order to fill out the tumor bed, it is brought into shape there by inserting a suitable filling medium. However, it is difficult to set the shape and the position of such a balloon catheter in a tumor bed in a precise fashion: the radiation dose decreases sharply as the distance from an X-ray radiation source increases. Hence the isocenter of a point X-ray radiation source in a balloon catheter should lie precisely in the middle of the balloon catheter and, at the same time, in the center of the tumor bed for a homogeneous irradiation of the tumor bed.

Since the location irradiated by the balloon catheter is within the patient, it is largely invisible to an operator. Simple positioning under direct viewing is therefore not possible.

Different methods are known for establishing the position of a balloon catheter with an X-ray radiation source: the position of catheter and X-ray source can be visualized on a display using imaging methods as well as computed tomography (CT) or ultrasound. However, this is very complex technically. Recording corresponding CT or ultrasound data also requires a comparatively long time. So as to be able to visualize an applicator by CT, X-ray radiation absorbing materials need to be used. Apart from the radiation load on healthy tissue caused by a CT recording due to the principles thereof, an applicator made of an X-ray radiation absorbing material has the following undesired side effect: this material also absorbs the therapeutic X-ray radiation during therapy. In order to compensate for this effect, the power of the X-ray source must then be increased or the irradiation time must be increased.

X-ray probes with a beryllium tip are often used in radiation therapy. Beryllium is a material that is almost transparent to X-ray radiation. Hence, it is difficult to see such X-ray probes in a CT image.

The X-ray probe from the INTRABEAM® radiation therapy system from Carl Zeiss allows minimally invasive access to a tumor bed in a patient due to the great length of 10 cm and the small external diameter of only 3.2 mm. In this X-ray probe geometry, elastic and plastic deformations of the X-ray probe due to lateral forces exerted thereon have to be accepted.

The X-ray probe from the INTRABEAM® radiation therapy system is designed as an evacuated electron beam tube. A beam of accelerated electrons is generated in this electron beam tube. The electron beam is directed at a gold target. There the electrons are decelerated abruptly and X-ray bremsstrahlung is created.

Since the electron beam tube in the INTRABEAM® radiation therapy system is very thin with a diameter of only 3.2 mm, the X-ray tube is very sensitive in respect of mechanical loads: this is because if the X-ray probe is bent, the electron beam in the electron beam tube no longer hits the target once a certain deflection has been reached. The result of this is that X-ray beams then are no longer generated, or only generated in an undefined fashion. In order to compensate for bending of the X-ray probe within certain limits, magnetic deflection coils are assigned to the electron beam tube in the INTRABEAM® radiation therapy system. Suitable actuation of these deflection coils using a system controller allows movement of the electron beam on the gold target of the order of +/−0.5 mm.

A safety mechanism is integrated into the INTRABEAM® radiation therapy system and it ensures that the X-ray radiation source is switched off if the intensity of the generated X-ray radiation drops below a certain threshold due to bending of the X-ray probe. Provision is made for the system in that case to be newly calibrated or verified in order to continue the therapy so as to be able to plan and apply the remaining dose to a patient. A patient undergoing therapeutic treatment may have to be sedated for an increased period of time because of this, but this harbors corresponding risks.

Hence, it is necessary to verify before each therapeutic use of the INTRABEAM® radiation therapy system that the X-ray probe of the system is not bent.

The X-ray probe is then inserted into a channel provided for it in a catheter for use in the patient. Since the course of a biopsy channel within a patient cannot, in general, readily have an exactly straight embodiment, there is the risk of the X-ray probe being subjected to mechanical forces that easily bend it when the corresponding X-ray probe is inserted into the catheter. However, such forces can occur not only during the insertion of a corresponding X-ray probe into a catheter. There can also be a mechanical load exerted on the corresponding X-ray probe during radiation therapy undergone by the patient, for example due to the respiratory movement of the patient.

The radiation field of an X-ray probe is determined by the spatial emission characteristic thereof. Isotropic point sources are desirable as X-ray radiation sources in IORT. Isotropic point sources are sources characterized by equidistant isodose lines from the center of the corresponding sources. Such radiation sources are therefore particularly suitable for tumor irradiation because the target area for IORT irradiation is usually spherical. An equal distance between the tissue surrounding the balloon of the catheter and the radiation source can be ensured by irradiating body tissue by means of an X-ray probe arranged in a balloon catheter. If a source that is not an ideal point source is used as a source for therapeutic irradiation, it is necessary to set a desired local radiation dose for the target area by treatment planning.

In particular applications or tumor positions within the body it is necessary to protect tissue and structures such as e.g. skin or nerves from being irradiated. Radiation planning can only partly take this aspect into account. In order to protect certain tissue structures in the human body from radiation damage, sources of therapeutic radiation are therefore operated with appropriate shielding apparatuses for therapeutic radiation: in the case of balloon catheters for radiation sources, the provision of materials with strong radiation-absorbing properties, e.g. lead or tungsten for the balloon wall or a balloon layer on the balloon catheter, is known. It is also known to fill the balloon of the balloon catheter with fluid media that absorb therapeutic radiation, e.g. a BaSO₄ solution.

Radiation from a point radiation source can be attenuated in an even and homogeneous fashion by providing lead or tungsten in the wall of the balloon in a balloon catheter or by filling the balloon of the catheter with a therapeutic-radiation absorbing medium. However, if a non-centrosymmetric radiation profile should be set for a point radiation source, provision can be made for a balloon catheter with segments or fill chambers containing material that absorbs radiation. Such balloon catheters are known in the art. However, they can only be produced with high production complexity, which becomes ever higher as the intended irradiation dose for certain tissue structures is set more finely in space.

It is also known to place covers made of a shielding material onto the outer side of a balloon catheter or else of a rigid applicator. By way of example, these covers can be pre-embossed films. Such films allow simple or else complex shielding on corresponding applicators. Such films can also be cut manually for a certain, concrete therapeutic application. However, this measure harbors the risk of the shielding made of film slipping in a patient's body during IORT or even remaining within the patient after the applicator has been extracted from the patient.

Nor is using covers made of shielding material indicated by the aspect of it being advantageous for therapeutic radiation to be applied through a biopsy channel by using a balloon catheter. However, then there is no fluid medium in the balloon when a corresponding balloon catheter is inserted into the biopsy channel. Rather, the balloon is in its smallest packaging size. Then material shielding therapeutic radiation cannot be used in the form of films.

Applicators are used in IORT for irradiating tumors and these act as a type of placeholder because the tumor bed would otherwise collapse onto itself. The tumor bed is widened by means of an applicator in order to ensure irradiation that is as homogeneous as possible of the remaining wound cavity. The applicator allows access to the irradiation location within the patient. By way of example, suitable applicators can be designed as flexible balloon catheters. Since corresponding applicators are in situ during the irradiation, they influence the radiation dose applied to the patient if they absorb or scatter X-ray radiation. This influence has to be considered during treatment planning. The relevant applicator data, more particularly the depth-dose curves, are therefore generally stored on a computer for the treatment planning.

When the applicator for irradiation is connected or adapted to a corresponding system, the presence of an applicator has been detected in systems corresponding to the prior art, but information relating to the applicator, such as the radiological data thereof, and its type, size and batch or serial number, is not registered. Hence, in practice there is the risk of the selected applicator not corresponding to the treatment plan. Applicators past their sterility expiry date are even used from time to time. This can lead to a too low or too high radiation dose applied to the tissue, or even to an infection.

When a new applicator is delivered, the data thereof is advantageously stored separately on suitable storage media, which are also delivered or which are available on servers in the form of files to be downloaded onto a customer computer. This affords the possibility of automatically using this data for a treatment plan when using a computer.

The radiation therapy system must be provided with the applicator number, type and size of an applicator selected for irradiation. This can be entered manually by using a keyboard. However, automatic registration of this information by means of an external scanner is less susceptible to errors. In this case, the identification is brought about by means of e.g. a barcode located on the sterile packaging or on the applicator itself. However, a barcode on the packaging of the applicator or on the applicator itself does not in all cases ensure that the applicator used is in fact the registered applicator. This is because in principle the applicator could be exchanged after registration. This can have fatal consequences because a satisfactory irradiation in respect of the applying of the desired dose is no longer possible in a controlled fashion.

An object of the present invention is to confront the aforementioned problems. This object is achieved by a balloon catheter with the features of claim 1 and by an X-ray applicator with the features of claims 8, 13 and 14.

The balloon catheter has a balloon, which can be filled by a medium and whose volume can increase, and a catheter shaft for inserting the X-ray applicator. The balloon or the catheter shaft has a stiff inner end piece in the continuation of the catheter shaft. The stiff end piece can consist of a plastic or another material largely transparent to X-ray radiation, for example a material equivalent to water in respect of the X-ray radiation transparency.

The balloon itself advantageously consists of an elastic material such as silicone or another air and liquid tight material such as urethane or PET that can be inflated. The shape of the balloon in the inflated state, or in the state filled to capacity with liquid, is dimensionally stable and can be round such that the balloon filled to capacity has a spherical shape.

The catheter shaft expediently comprises a flexible, soft tube, which consists of e.g. silicone. This ensures a comfortable wear for a patient with the catheter inserted into their body: the catheter can then be placed tightly against the patient body, which minimizes the risk of mechanical load on a catheter inserted into a patient body, or the displacement of said catheter, as a result of impacts and catching on objects.

The end piece of the catheter shaft expediently is cylindrical and has a cylinder axis that runs substantially coaxially to the axis of the catheter shaft. The end piece advantageously has a round cross section perpendicular to the cylinder axis. However, the cross section can also be star-shaped. The cross section of the end piece is preferably perforated like a cage.

A mechanical stop is advantageously formed in the interior of the end piece. Alternatively, a mechanical stop for the X-ray probe can also be provided without an end piece in the interior of the balloon.

A filter that absorbs X-ray radiation is advantageously arranged in the interior of the end piece. This is particularly expedient if the material of the end piece has a lower X-ray radiation absorption than the material which fills the balloon. Alternatively, the end piece can also have a perforated design and be provided with openings such that the medium to be filled into the balloon in order to inflate the latter can enter the interior of the end piece or a part forming the mechanical stop.

In the region of the end piece or in the interior of the balloon, the balloon catheter advantageously has two partial cylinder shells made of an X-ray radiation absorbing material, which are arranged coaxially and rotatably with respect to one another. By rotating the partial cylinder shells relative to one another, it is possible to vary the solid angle through which the X-ray radiation can be emitted to the surrounding tissue from the balloon catheter.

The invention furthermore relates to an X-ray applicator for use with a balloon catheter having one or more of the above-described properties.

The X-ray applicator can additionally comprise an X-ray probe with an evacuated tube and with a target arranged therein and an electron source and an electron accelerator.

The X-ray applicator can moreover have a probe protection apparatus, which contains a stable tube for inserting the X-ray probe, and can be connected to the flexible part of the catheter shaft by means of an interface. The probe protection apparatus can be separated from the X-ray applicator and can be connected to the latter. This affords the possibility of stiffening the balloon catheter for the duration of the irradiation by connecting balloon catheter and applicator.

The probe protection apparatus can have an encodement in the region of the interface, which encodement interacts with sensors on a different part of the X-ray applicator. By way of example, the encodement can comprise a bar code.

The invention also relates to a modular arrangement with X-ray applicator, comprising:
a) a base unit with an electron source, an electron accelerator, an evacuated tube and, arranged at a distal end in the evacuated tube, a target for generating X-ray radiation by electrons impinging on the target,
b) a probe protection apparatus, which can be held on the base unit and separated from the latter and which has a tube into which the evacuated tube of the base unit can be inserted, and
c) a balloon catheter, which has a proximal, flexible tube, a distal, stiff end piece and a balloon whose volume can increase.

The invention also relates to a modular arrangement with X-ray applicator, comprising:
a) a base unit with an electron source, an electron accelerator, an evacuated tube and, arranged at a distal end in the evacuated tube, a target for generating X-ray radiation by electrons impinging on the target, and
b) a probe protection apparatus, which can be held on the base unit and separated from the latter and which has a tube into which the evacuated tube of the base unit can be inserted, wherein the probe protection apparatus has an encodement in the region of the interface to the base unit.

Details of the invention will be explained in more detail in the following text using the exemplary embodiments illustrated in the figures, in which:

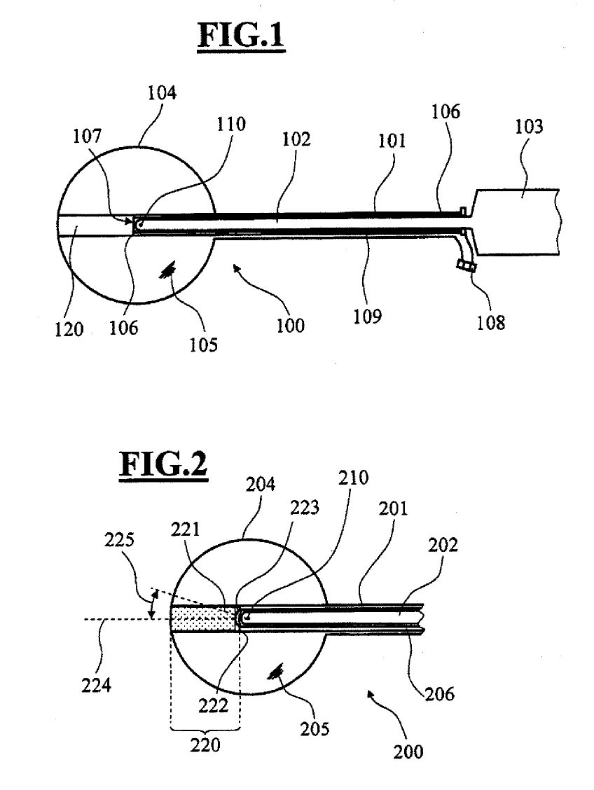

FIG. 1 shows a section of a first exemplary embodiment of a balloon catheter with X-ray applicator;

FIG. 2 shows a partial section of a second exemplary embodiment of a balloon catheter with an X-ray probe inserted therein;

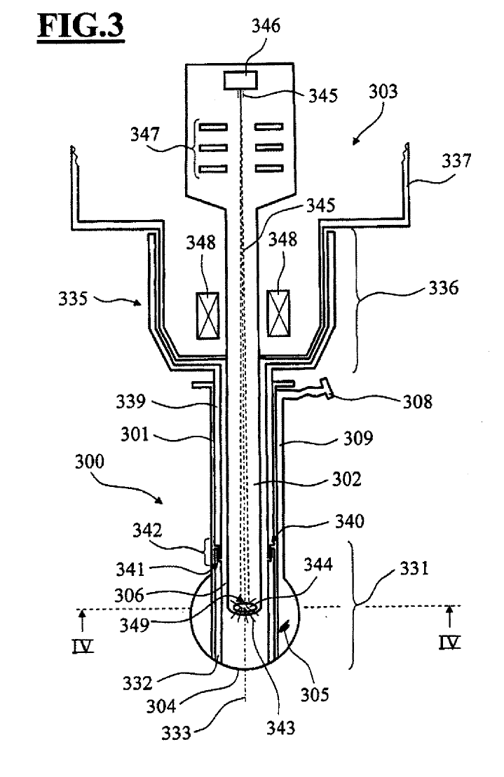

FIG. 3 shows a third exemplary embodiment of a balloon catheter with an X-ray applicator, which has been assigned a first embodiment of a probe protection;

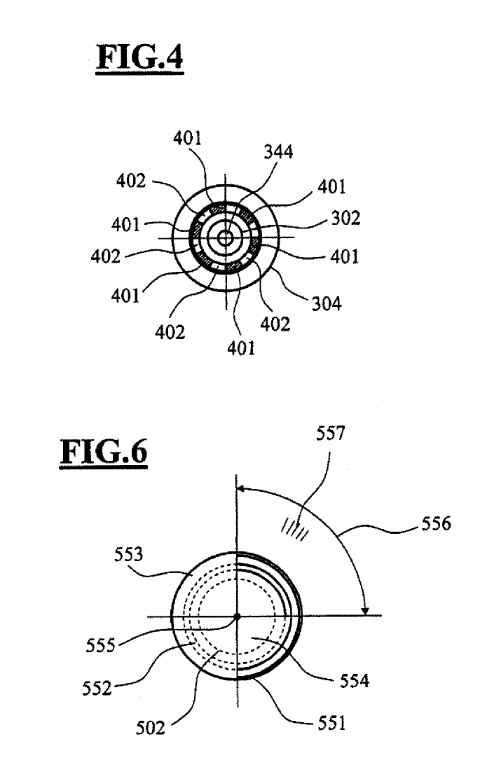

FIG. 4 shows a section of the X-ray applicator with balloon catheter;

FIG. 5 shows an X-ray applicator with a second alternative embodiment for a probe protection device;

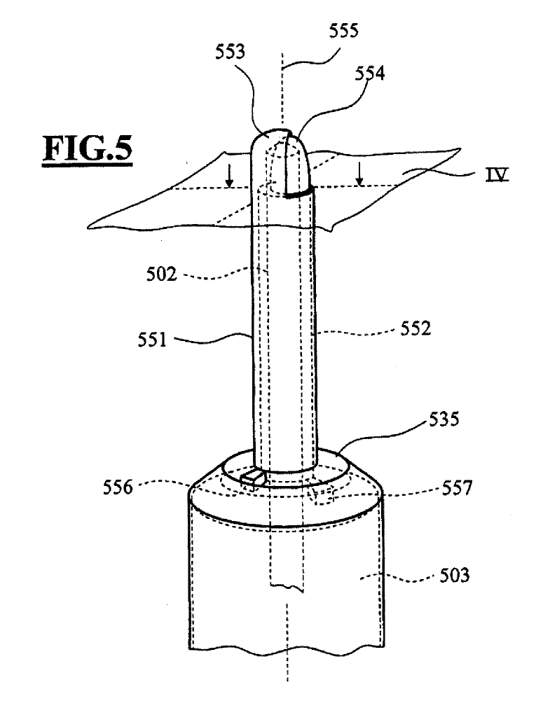

FIG. 6 shows a section in the plane VI from FIG. 5;

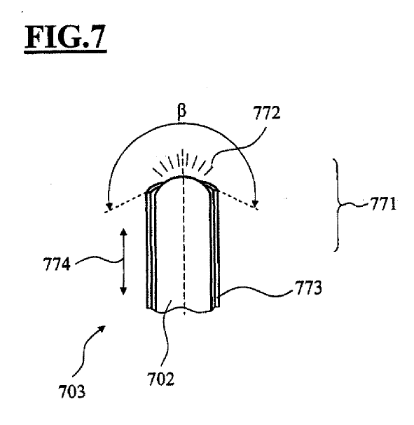

FIG. 7 shows a section of the X-ray probe of an X-ray applicator and a section of a third alternative embodiment for a probe protection device;

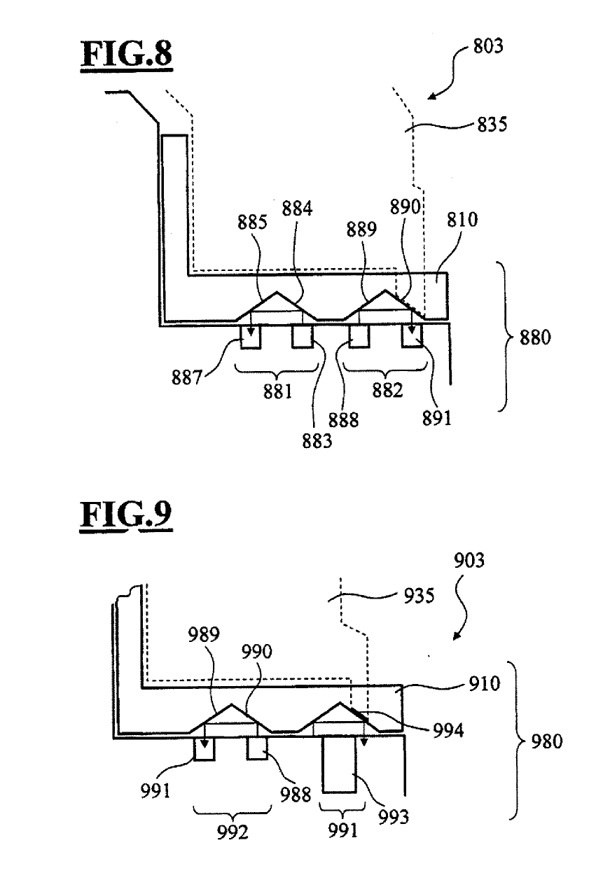

FIG. 8 shows a first embodiment for a connection between X-ray applicator and probe protection; and

FIG. 9 shows a second embodiment for a connection between X-ray applicator and probe protection.

FIG. 1 shows a balloon catheter 100 with a catheter shaft 101, into which an X-ray probe 102 of an X-ray applicator 103 has been inserted. The balloon catheter comprises a balloon 104 filled with a fluid medium 105. The catheter shaft 101 consists of a flexible plastics material. A lumen 106 is formed in the catheter shaft 101. A mechanical stop 107 at an inner end piece 120 located in the balloon 104 is provided in the balloon 104. The stop 107 allows simple and quick positioning of the X-ray probe 102 in the balloon catheter. The X-ray probe 102 can be inserted into the balloon 104 through the catheter shaft 101. The balloon 104 is made of a hard plastics, e.g. PET. By contrast, the catheter shaft 101 consists of a soft and elastic plastics, e.g. silicone.

A connection 108 is formed on the balloon catheter 100. The connection 108 is connected to the balloon 104 of the balloon catheter 100 via a fluid line 109. The balloon 104 can be filled with the fluid medium 105 via the connection 108. A sterile isotonic saline solution is particularly suitable as a fluid medium 105. A sterile isotonic saline solution ensures high patient safety. However, in principle gases may also be used for filling the balloon 104 of the balloon catheter.

The X-ray probe is in the inner lumen 106 of the catheter shaft 101 for operation in the balloon catheter 100. There said probe is in direct contact with the stop 107. As a result, the isocenter of the X-ray probe 102, i.e. the center of the region from which the X-ray radiation emanates, can be arranged in the center 110 of the balloon 104 of the balloon catheter 100. Additionally, this can avoid unnecessary radiation exposure of healthy tissue in the patient, which exposure would arise from imaging methods such as CT for gauging whether the X-ray probe was correctly arranged in the balloon catheter.

The stop 107 material has similar radiation-physical properties as an isotonic saline solution, which is suitable for use as a filling medium 105 for the balloon 104. The effect of this is that an isotropic radiation field generated by the X-ray probe is not influenced strongly by the balloon catheter, as it would be in case of different X-ray radiation scattering properties between filling medium and stop material in the balloon catheter. The position of the stop 107 in the balloon 104 of the balloon catheter 100 is matched to the geometry of the X-ray probe 102 as follows: when the arrangement is in operation, the X-ray probe 102 isocenter, i.e. the center of the region emanating X-ray radiation, is in the center 110 of the balloon catheter 100. Moreover, this measure ensures that the patient is not subjected to an excessive radiation load for visualizing the position of the X-ray probe 102 of the X-ray applicator 103 when the latter in the balloon catheter 100 is inserted into a patient body.

A material that has a scattering characteristic for X-ray radiation that differs from the scattering characteristic of the fluid medium 105 used to fill the balloon 104 of the balloon catheter 100 can also be provided for the stop 107 in the balloon catheter 100. If the stop 107 consists of a material that strongly absorbs or scatters X-ray radiation, a suitable geometry of the stop 107 allows the latter to have little negative influence on the emission characteristic of the X-ray probe 102. By way of example, if the stop is formed with a star-shaped, hollow-cylindrical or cage-like geometry, there are openings on the stop through which X-ray radiation can pass through without hindrance.

Suitable parts of the balloon catheter 100 can be doped with material that scatters X-ray radiation in order to set an isotropic radiation field for the X-ray radiation generated by the X-ray probe 102. Alternatively, or in addition thereto, shielding and filters can be provided in the balloon catheter 100.

FIG. 2 shows a partial section of a balloon catheter 200 with an X-ray probe 202, which balloon catheter has been modified with respect to the balloon catheter 100 in FIG. 1. To the extent that components in the section 200 of the balloon catheter correspond to components found in the balloon catheter 100 from FIG. 1, these have been identified in FIG. 2 by reference signs in the form of numbers that have been increased by 100 compared to FIG. 1.

In the balloon catheter in FIG. 2, a stop 221 for the X-ray probe 202 is formed in a section 220 acting as an end piece in the balloon 204 in the catheter shaft 201 housing the X-ray probe 202. This stop 221 consists of a material that damps or scatters X-ray radiation to a smaller degree than the filling medium 205 provided for the balloon 204.

There is a filter 223 on the end face 222 of the stop 221 facing the X-ray probe 202. This filter 223 consists of aluminum. Aluminum has comparatively high X-ray radiation absorption. In the sectional plane of the partial section shown in FIG. 2, the filter 223 has a crescent-shaped cross section. The effect of this filter 223 geometry is that X-ray radiation emitted by the X-ray probe 202 in the direction of the axis 2204 is attenuated more strongly than X-ray radiation emitted by the X-ray probe 202 at an angle 225 with respect to the axis 224.

It should be noted that another substance such as tungsten or barium can also be used as filter 223 material instead of aluminum. The stop 221 itself can also be made of a material that absorbs X-ray radiation, e.g. plastics doped with substances that strongly absorb or scatter X-ray radiation.

FIG. 3 shows a section of a further balloon catheter 300 with X-ray applicator 303. The balloon catheter 300 has been modified with respect to the balloon catheter 100 from FIG. 1 and the balloon catheter 200 from FIG. 2. To the extent that the balloon catheter 300 and the X-ray applicator 303 have components that are also provided in the balloon catheter 100 and X-ray applicator 103 from FIG. 1, these have reference signs in FIG. 3 in the form of numbers that have been increased by the number 200 compared to FIG. 1.

The balloon catheter 300 has a catheter shaft 301 with a lumen 306 for holding the X-ray probe 302 of the X-ray applicator 303. The balloon catheter 300 comprises a balloon 304 arranged at a front section 331 of the catheter shaft 301. A fluid line 309 to a connection 308 is formed in the balloon catheter 300. The balloon 304 can be filled with a fluid medium 305 via the connection 308. An end piece in the form of a tubular stabilizing element 332 is arranged in the front section 331 of the balloon catheter 300. The tubular stabilizing element 332 consists of plastics. This tubular stabilizing element 332 has a dual function: firstly, it stiffens the balloon catheter 300 in the front section in the direction of the axis 333; secondly, it serves as a stop for the sleeve-shaped attachment 334 of a probe protection apparatus 335. The sleeve-shaped attachment 339 consists of stiff plastic. However, the sleeve-shaped attachment 339 can also be embodied in stainless steel. The sleeve-shaped attachment 339 has a tubular design. It stabilizes the X-ray probe 302. The probe protection device 335 is connected to the X-ray applicator 303 by a first interface 336 fixedly connected to the housing 337 of the X-ray applicator 303.

The probe protection apparatus 335 has an end section 340 in the sleeve-shaped attachment 339. This end section 340 is designed to engage into a reception section 341 found in the tubular stabilizing element 332. The end section 340 of the sleeve-shaped attachment 339 of the probe protection apparatus 335 and the reception section 341 of the tubular stabilizing element 332 thus form a second interface 342 that acts as a frictional connection.

The balloon catheter 300 is designed for holding the X-ray probe 302 with the sleeve-shaped attachment 339 of the probe protection apparatus 335.

In the process, the geometry of the probe protection apparatus 335 with the interfaces 336 and 342 is matched to the geometry of the X-ray applicator 303 around the balloon catheter 300 such that, taken individually, the emission center for X-ray radiation 343 from the X-ray probe 302 is located in the center of the balloon 304 when the latter is filled to capacity with fluid medium 305.

In order to generate X-ray radiation 343, a target 344 consisting of gold is arranged in the X-ray probe 302. Electrons 345 from an electron source 346 are accelerated toward this target 344 by means of high voltage applied to an acceleration stage 347. The X-ray applicator 303 contains magnetic deflection coils 348. The magnetic deflection coils 348 can be used to adjust a magnetic field for deflecting the electrons 345 accelerated toward the target 344. This affords the possibility of adjusting the site 349 at which the accelerated electrons 345 are incident on the target. This allows adjustment of the spatial radiation profile of the X-ray radiation 343 emitted by the X-ray probe 302, and changes in the spatial radiation profile due to bending in the X-ray probe 302 can be compensated for to a certain extent.

The sleeve-shaped attachment 339 of the probe protection apparatus 335 acts as a mechanical stabilizer for the X-ray probe 302. It secures the X-ray probe 302 against bending with respect to the axis 333. This measure allows the introduction of mechanical forces into the X-ray applicator 303 through the balloon 304 of the balloon catheter 300 and the sleeve-shaped attachment 339 of the probe protection apparatus 335 without there being excessive mechanical loads on the X-ray probe such 302, which affect the radiation profile of the X-ray radiation emitted by the X-ray probe such that the radiation can no longer be compensated for by suitably actuating the magnetic deflection coils 348.

Hence, the arrangement of X-ray applicator 303, balloon catheter 300 and probe protection apparatus 335 shown in FIG. 3 is particularly suitable for use in an adjustable support device, which is automatically adjusted and tracked as a result of the forces introduced into the arrangement in order, for example, to compensate for respiratory movements of a patient in IORT.

Such an adjustable support device can for example be embodied as a server apparatus, in which the support axes are adjusted by means of suitable actuators as a result of a force introduced into the arrangement of X-ray applicator 303, balloon catheter 300 and probe protection apparatus 335. However, a support device can also be provided as a support device with a balanced support axis, in which the force absorbed by the arrangement of X-ray applicator 303, balloon catheter 300 and probe protection apparatus 335 overcomes friction and inertia forces that occur on the corresponding support.

FIG. 4 shows a section of the balloon catheter 300 with probe protection apparatus 335 and X-ray applicator 303 from FIG. 3 along the line IV-IV. To the extent that FIG. 4 shows components that can also be seen in FIG. 3, these components are identified by the same reference signs as in FIG. 3.

In the region of the intended operating position of the X-ray probe 302 in the balloon 304 of the balloon catheter, the wall 401 of the tubular stabilizing element 332 of the probe protection apparatus has perforations 402 through which the X-ray radiation can penetrate the patient tissue in an undamped fashion via the balloon 304.

It should be noted that, as an alternative to the arrangements of balloon catheter and X-ray applicators explained in FIGS. 1, 2, 3 and 4, provision can also be made for the balloon catheter to be formed without a corresponding stop for the X-ray probe, or that provision can be made for a probe protection apparatus that allows free positioning of the X-ray probe in the balloon catheter. In the process, it is expedient for a mechanical or else an electrical drive to be provided for moving the X-ray applicator in the balloon catheter.

FIG. 5 shows an X-ray applicator 503 with a probe protection apparatus 535, which applicator is suitable for IORT with a balloon catheter, as has been described on the basis of FIGS. 1, 2, 3 and 4. To the extent that the X-ray applicator 503 has components that correspond to components of the X-ray applicator 303 from FIG. 3, these components are identified by numbers as reference signs that have been increased by the number 100 in comparison with FIG. 3.

The X-ray applicator 503 comprises an X-ray probe 502. The X-ray probe 502 is in a probe protection apparatus 535. The probe protection apparatus 535 comprises a first sleeve-shaped attachment 551 and a second sleeve-shaped attachment 552. A first hemispherical end 553 is formed at the distal end of the first sleeve-shaped attachment 551. The second sleeve-shaped attachment 552 has a hemispherical end 554. The hemispherical ends 553, 554 have the shape of partial cylinder shells.

The first hemispherical end 553 and the second hemispherical end 554 consist of stainless steel. Stainless steel is a material that strongly absorbs X-ray radiation.

The second sleeve-shaped attachment 552 can be rotated about the axis 555 in the first sleeve-shaped attachment 551.

The probe protection apparatus 535 is provided with an electrical drive 556 for rotating the first sleeve-shaped attachment 551. The second sleeve-shaped attachment 552 can be moved about the axis 555 by means of an electrical drive 557. It is possible to rotate the hemispherical ends 553, 554 coaxially with respect to one another by adjusting the first sleeve-shaped attachment 551 and the second sleeve-shaped attachment 552.

FIG. 6 shows a section of the X-ray applicator 503 with the probe protection apparatus 535 in the sectional plane identified by VI in FIG. 5 and the viewing direction indicated therein. The same components are identified by identical reference signs in FIG. 5 and FIG. 6.

By adjusting the hemispherical ends 553, 554 relative to one another about the axis 555, it is possible to set an aperture angle 556 over which X-ray radiation 557 for IORT can be emitted to patient tissue from the X-ray probe 502 in the corresponding balloon catheter.

The movable hemispherical ends 553, 554 thus allow the defined configuration of the arrangement for a radiation therapy application. It should be noted that mechanical drives can also be provided for adjusting the hemispherical ends 553, 554 instead of two electrical drives 556, 557. Moreover, it is possible to provide only one drive and to couple the two sleeve-shaped attachments to one another by means of a transmission such that said attachments can be moved toward one another in a coordinated fashion.

FIG. 7 shows a section of a further modified embodiment of an X-ray applicator with a probe protection apparatus, which embodiment is suitable for use with a balloon catheter. The X-ray applicator 703 has an X-ray probe 702 that provides X-ray radiation 772 with an isocentric emission characteristic in a front section 771. The probe protection apparatus is designed with an adjustable sleeve-shaped section 773, which consists of stainless steel and thus strongly absorbs X-ray radiation. By moving the sleeve-shaped section 773 in the region of the front section in accordance with the double-headed arrow 774, it is possible to vary the solid angle region “p” over which X-ray radiation in a balloon catheter is dispensed to patient tissue. Within the scope of a further modified embodiment, the principle for setting the probe protection apparatus in the case of the X-ray applicator described on the basis of FIG. 5 and FIG. 6 can be combined with the principle for setting the probe protection apparatus in the case of the X-ray applicator described on the basis of FIG. 7: by providing the movable hemispherical ends in the X-ray applicator according to FIG. 5 and FIG. 6 and the linearly movable sleeve in accordance with the X-ray applicator according to FIG. 7, a radiation field generated by an appropriate X-ray probe can be set in an even more precise fashion.

It should be noted that an appropriate X-ray applicator need not necessarily be provided with hemispherical ends only. Rather, corresponding ends may also be embodied to be straight, oblique or oval. This allows an aperture window for X-ray radiation to be set independently of one another in two directions. More particularly, this affords the possibility of setting the solid angle segment into which X-ray radiation is emitted from the X-ray probe in a defined fashion.

FIG. 8 shows a section of a further X-ray applicator 803, which has been assigned a probe protection apparatus 835.

Systems emitting therapeutic X-ray radiation can harm the surroundings, operating staff and patients in the case of incorrect operation. In order to ensure high operational safety, the arrangement of X-ray applicator 803 and probe protection apparatus 835 shown in FIG. 8 comprises an interlock system 880. The interlock system 880 has a connection section 810 by means of which it can be attached to a support apparatus (not illustrated in any more detail). The interlock system 880 is fixedly connected to the probe protection apparatus 835. The interlock system 880 comprises a first unit 881 for interlocking and a corresponding second unit 882. The first unit 881 has a transmitter 883 that generates a first optical signal, which is supplied to a receiver unit 887 via mirrored surfaces 884, 885 on the interlock system 880.

The second unit 882 has a transmitter 888, which generates a corresponding pulsed optical signal, which can reach a receiver unit 891 over mirrored surfaces 889, 890. The optical signals from the first unit 881 and the second unit 882 have different pulse frequencies.

The interlock system 880 is connected to a control unit of the X-ray applicator (not illustrated in any more detail). It ensures that the X-ray applicator 803 emits X-ray radiation only if the probe protection apparatus 835 is connected to the X-ray applicator 803.

FIG. 9 shows a further X-ray applicator 903 with a probe protection apparatus 935. This arrangement comprises an interlock system 980 with a connection section 990. This connection section 990 allows the X-ray applicator 903 and the probe protection apparatus 935 in turn to be housed in a support apparatus (not illustrated in any more detail).

As a first unit 991, the interlock system 980 contains a barcode reader 993 designed to read out encrypted data. The barcode reader 993 can be used to read out a barcode 994 as an encodement in the connection section 990. A unit corresponding to the first unit in the interlock system 880 from FIG. 8 is provided as second unit 992 in the interlock system. The components of this second unit 992 can be identified by numbers as reference signs that have been increased by the number 100 in comparison with FIG. 8.

This data is transferred into the system and thus allows appropriate treatment planning. Thus, the system can prevent irradiation if certain basic conditions, e.g. size, type, expiry date, are not satisfied. In this case, it is 100% certain that the planned applicator is also actually used for the irradiation. It is likewise possible for the barcode only to contain a factor or a function, as a result of which a standard file located on the system for this applicator type is matched to the actually adapted applicator.

The barcode reader can be integrated in the optical interlock or attached as an additional barcode reader.

The barcode can be applied directly on the reflective surface or on the shaft of the applicator and/or that of the X-ray probe protection.

1-16. (canceled)

17. A balloon catheter, comprising:
a balloon having an interior volume that is variable, the interior of the balloon being configured to be filled by a medium; and
a catheter shaft configured so that an X-ray probe can be inserted into the catheter shaft, a portion of the catheter shaft being partially disposed in the interior of the balloon,
wherein:
an element of the balloon catheter has a stiff inner end piece that is contiguous with the catheter shaft; and
the element comprises the balloon or the catheter shaft.

18. The balloon catheter of claim 17, wherein the catheter shaft comprises a flexible, soft tube.

19. The balloon catheter of claim 17, wherein the end piece is cylindrical, and the end piece has a cylinder axis that runs substantially coaxially with an axis of the catheter shaft.

20. The balloon catheter of claim 19, wherein the end piece has a round or star-shaped cross section perpendicular to the cylinder axis.

21. The balloon catheter of claim 20, wherein the cross section of the end piece is perforated.

22. The balloon catheter of claim 20, wherein the cross section of the end piece is cage-like.

23. The balloon catheter of claim 17, further comprising a mechanical stop in an interior of the end piece.

24. The balloon catheter of claim 17, further comprising a filter in an interior of the end piece.

25. The balloon catheter of claim 17, further comprising an X-ray radiation absorbing material.

26. The balloon catheter of claim 17, further comprising two partial cylinder shells comprising an X-ray radiation absorbing material, the two partial cylinder shells being coaxial and rotatable with respect to each other.

27. A system, comprising:
the balloon catheter of claim 17; and
an X-ray applicator.

28. The system of claim 27, further comprising a target, an electron source, and an electron accelerator.

29. The system of claim 27, further comprising a probe protection apparatus which comprises a tube configured so that an X-ray probe can be inserted therein.

30. The system of claim 29, wherein the probe protection apparatus can be separated from the X-ray applicator, and the probe protection can be connected to the X-ray applicator.

31. The system of claim 29, wherein the probe protection apparatus comprises two partial cylinder shells comprising an X-ray radiation absorbing material, and the two partial cylindrical shells are rotatable coaxially with respect to each other.

32. The system of claim 29, wherein the probe protection apparatus is encoded.

33. The system of claim 32, wherein the probe protection unit is encoded in a region of an interface between the probe protection unit and the base unit.

34. A system, comprising:
a base unit, comprising:
an electron source;
an electron accelerator;
an evacuated tube; and
a target in the evacuated tube at a distal end of the evacuated tube, the target being capable of generating X-ray radiation when impinged by electrons;
a probe protection apparatus capable of being connected to the base unit, the probe protection apparatus capable of being separated from the base unit; and the probe protection apparatus comprising a tube into which the evacuated tube of the base unit can be inserted; and
a balloon catheter comprising:
a balloon having an interior volume that is variable;
a proximal, flexible tube; and
a distal, stiff end piece.

35. A system, comprising:
a base unit, comprising:
an electron source;
an electron accelerator;
an evacuated tube; and
a target in the evacuated tube at a distal end of the evacuated tube, the target being capable of generating X-ray radiation when impinged by electrons; and
a probe protection apparatus capable of being connected to the base unit, the probe protection apparatus capable of being separated from the base unit; and the probe protection apparatus comprising a tube into which the evacuated tube of the base unit can be inserted;
wherein the probe protection apparatus is encoded.

36. The system of claim 35, wherein the probe protection unit is encoded in a region of an interface between the probe protection unit and the base unit.
